(19) United States
(12) Patent Application Publication
(10) Pub. No.: US 2022/0379092 A1
(43) Pub. Date: Dec. 1, 2022

(54) CATHETER COVERING DEVICE

(71) Applicant: Pascal Dabel, Los Angeles, CA (US)

(72) Inventor: Pascal Dabel, Los Angeles, CA (US)

(21) Appl. No.: 17/754,978

(22) PCT Filed: Feb. 13, 2020
(86) PCT No.: PCT/US20/18165

§ 371 Date: Apr. 18, 2022

(51) Int. Cl. A61M 25/02

(52) U.S. Cl. CPC A61M 25/02; A61M 2025/0266; A61M 2205/15; A61M 2205/584; A61M 2025/0246

(57) ABSTRACT

This invention relates to a catheter cover. Previously, catheter covers that may protect a catheter from water were not efficiently securable to a human user. Embodiments of the present invention use a tab (50) joined to a sleeve on the catheter cover. A first adhesive strip (52) is joined to the tab and the sleeve and is configured to be attached to skin of a human user.

### TECHNICAL FIELD

The present invention relates to a central venous catheter cover and, more particularly, to a catheter cover that may protect the catheter from water and external contaminants.

### BACKGROUND ART

Healthcare patients that have to use a central venous catheter are not allowed to shower or swim for weeks and up to months at a time. The patients thereby become dirty, uncomfortable, and are more prone to infection. The only way a patient may clean themselves is by using a washcloth or sponge which promotes cross contamination of the catheter site. Further, the patients are at increased risk for blood stream infections since there are no appropriate methods of protecting the entire catheter and exit site.

Currently, to cover the catheter, gauze and tape may be used. However, the gauze only protects the exit site into the skin but are not water resistant. The gauze does not prevent contaminants from making contact with the catheter ports or exit site into the skin. The gauze and tape promotes absorption of moisture, dead skin cells and bacteria. All of the above increase the risk of infection instead of preventing it. Further, the gauze and tape fall off easily leaving little to no protection.

As can be seen, there is a need for a cover that protects the catheter from the elements and allows for an improvement in quality of life due to improved hygiene and lower risk of infection.

### DISCLOSURE OF THE INVENTION

A catheter covering device comprises a sleeve formed of a water resistant and breathable polyurethane and comprising a first end and a second end. The first end comprises a rim forming an opening into the sleeve. An adhesive film comprises a first side and a second side. The first side is attached to the rim of the sleeve and the second side comprises an adhesive. The adhesive film comprises an inner edge forming an opening leading into the sleeve.

A water and moisture indicator comprises a ring shape having a first side, a second side, an inner edge and an outer edge. The first side of the water indicator is attached to the second side of the adhesive film. An opening is formed by the inner edge aligns with the opening of the adhesive film and leads into the sleeve. An outer edge of the adhesive film extends beyond the outer edge of the water indicator so that the adhesive is exposed. A tab is joined to the sleeve.

A first adhesive strip is joined to the tab and the sleeve and configured to be attached to skin of a human user. A second adhesive strip is joined to the tab and the sleeve, and linearly aligned with the tab and the first adhesive strip and configured to be attached to the skin of the human user.

### BRIEF DESCRIPTION OF THE FIGURES

The detailed description of some embodiments of the invention is made below with reference to the accompanying figures, wherein like numerals represent corresponding parts of the figures.

FIG. 1 is a front view of an embodiment of the present invention shown in use;

FIG. 2 is a perspective view of an embodiment of the present invention;

FIG. 3 is a rear detail perspective view of an embodiment of the present invention shown with a wax paper;

FIG. 4 is a rear detail perspective view of an embodiment of the present invention demonstrating the removal of the wax paper of FIG. 3;

FIG. 5 is a front view of an embodiment of the present invention shown with a dry water indicator;

FIG. 6 is a front view of an embodiment of the present invention shown with a wet water indicator; and

FIG. 7 is a section detail view of an embodiment of the present invention along line 7-7 of FIG. 1.

### BEST MODE OF THE INVENTION

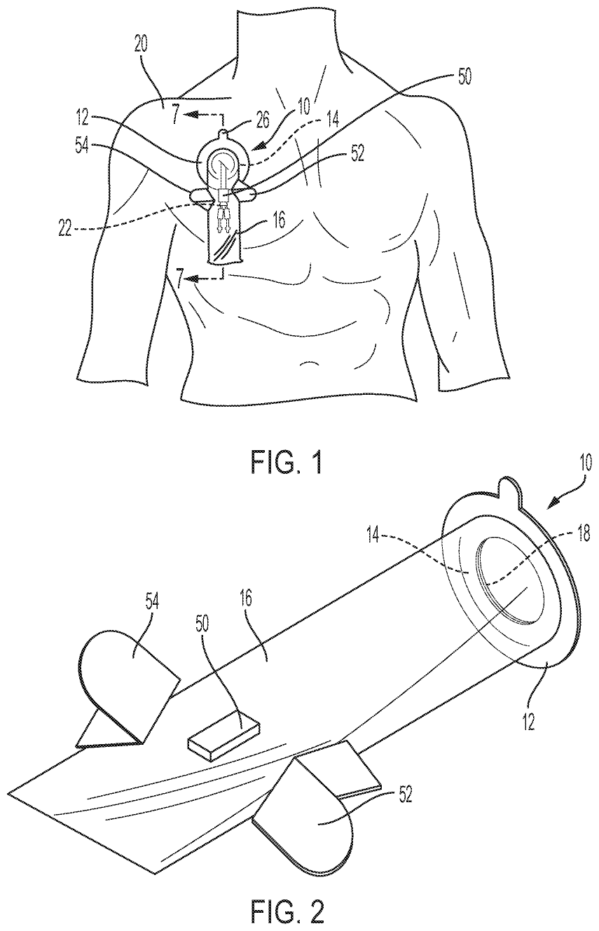

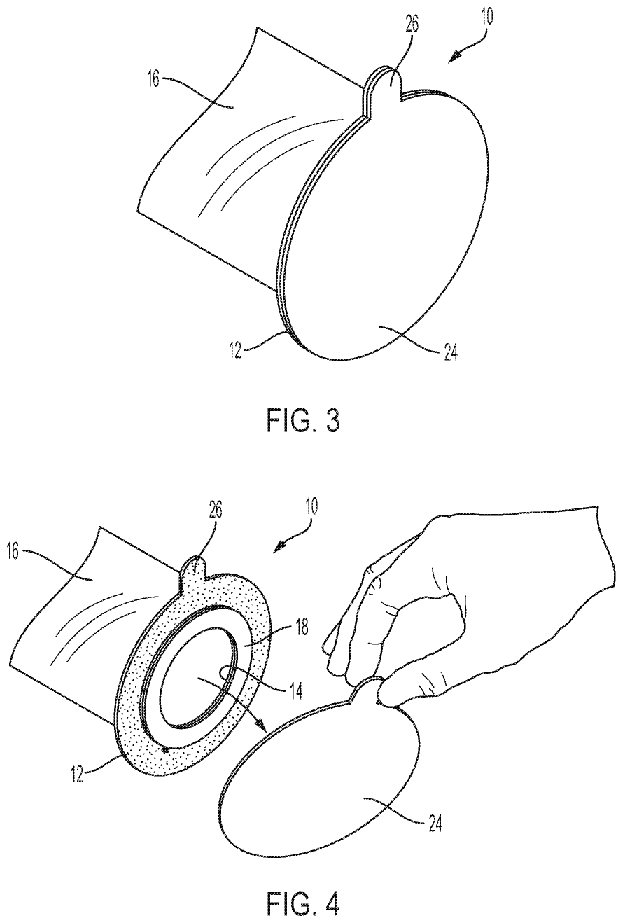

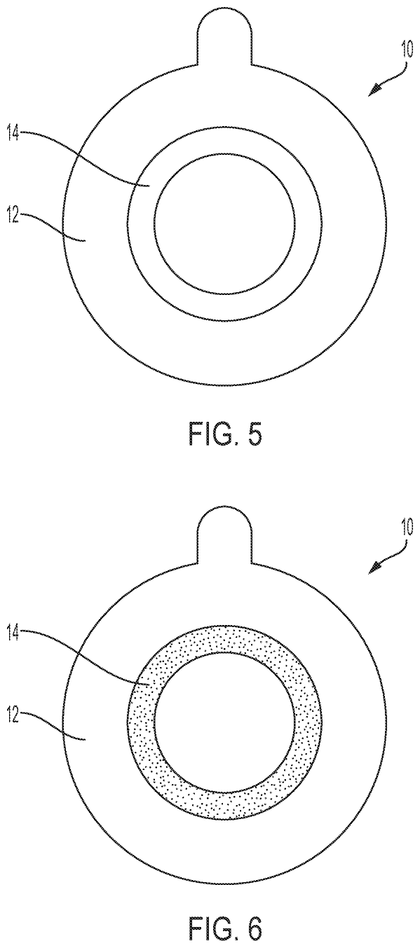

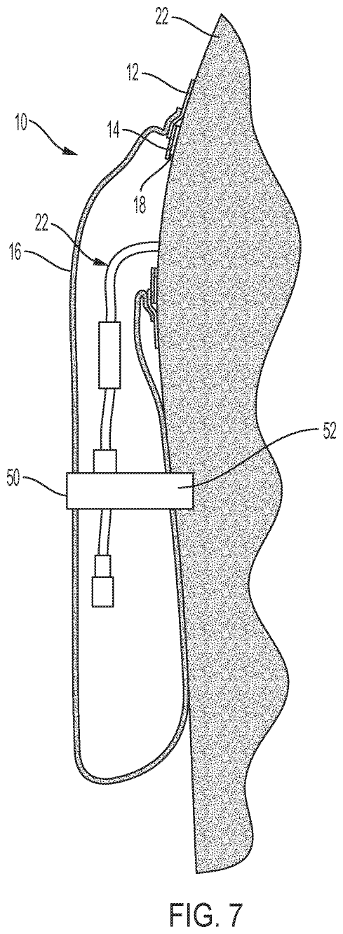

Referring to FIGS. 1 through 7, the present invention may include a catheter covering device 10. The catheter covering device 10 may include a water resistant sleeve 16. The water resistant sleeve 16 may include a first end and a second end. The first end may include a rim forming an opening into the sleeve 16. The second end may be a closed end. The water resistant sleeve 16 may be made of a polymer, such as a plastic. For example, the water resistant sleeve 16 may be made of an Ultra Violet protective polyethylene. In certain embodiments, the water resistant sleeve 16 may be approximately six to twelve inches long with about a one inch diameter. The sleeve 16 may be formed to fit over the catheter 22 of a user 20.

The present invention further includes an adhesive film 12. The adhesive film 12 is attached to the rim of the water resistant sleeve 16. The adhesive film 12 includes an opening that leads into the sleeve 16. In certain embodiments, the adhesive film 12 includes a first side and a second side. The rim of the sleeve 16 may be sealed, such as radiofrequency welded, to the first side of the adhesive film 12. The second side of the adhesive film 12 may include the adhesive material. The adhesive film 12 and the water resistant sleeve 16 may prevent water from contacting the catheter 22 and the skin surrounding the catheter 22. In certain embodiments, the adhesive film 12 is a waterproof tape, similar to 3M Tegaderm®. The adhesive film 12 may have a round shape, however the waterproof tape 12 may be any shape desired.

In certain embodiments, the present invention may include a water indicator 14. The water indicator 14 may be attached to the adhesive film 12. The water indicator 14 may be attached circumferentially around the opening of the adhesive film 12. In certain embodiments, the water indicator 14 may change color, from white to red, when moisture comes into contact with the water indicator 14. This indicates to the user 20 that moisture has entered the barrier and the user 20 may exit the water and remove the sleeve 16 and replace with a new device. The water indicator 14 may include 3M 5557® water indicator tape, an indigo color indicating paper, an electronic tape detector or the like.

The present invention may further include additional components. For example, the present invention may include a paper cover 24 and a tab 26. The paper cover 24 may be a wax paper that covers the adhesive when the present invention is not being used. The tab 26 may extend from the parameter of the adhesive film 12 and from the wax paper 24. The tab 26 may or may not include an adhesive material. Therefore, when the user 20 has the catheter covering device 10 attached to the skin, the user 20 may pull the tab 26 in order to remove the catheter covering device. Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

In some embodiments, a tab 50 is joined to the sleeve 16 proximate to the second end and on a side away from the human user. A first adhesive strip 52 is joined to the tab 50 and the sleeve 16 and configured to be attached to skin of a human user. A second adhesive strip 54 is joined to the tab 50 and the sleeve 16. The second adhesive strip 54 is linearly aligned with the tab 50 and the first adhesive strip 52 and is configured to be attached to the skin of the human user.

A method of using the present invention may include the following. First a user may provide the catheter covering device mentioned above. The user may groom an area of the body surrounding the catheter, which may include shaving and/or cleaning the area. The user may place the catheter through the opening and within the sleeve. The air may be squeezed out of the plastic sleeve. The rim of the water resistance sleeve may be adhered to the body by using waterproof tape around the catheter insertion site into the skin. The waterproof tape may be pressed firmly against dry and hairless skin around the catheter. Additionally, the first adhesive strip and the second adhesive strip can further secure the sleeve to the user. This may prevent water from getting into the device or making contact with the catheter in the shower or pool. If water were to accidentally get through the waterproof tape, the water may come into contact with the water indicator which may cause a color change, for example from white to red, blue to white or the like. The patient may then know to get out of the shower or pool and dry themselves off thoroughly without risk of separating the device because of the adhesive strips. The device may then be removed from the skin by pulling on the tab and discarded.

A method of making the present invention may include the following. Medical grade skin tape may be cut into circular shape. The color changing/water indicating tape may be cut with desiccant paper into a smaller circular shape, i.e. a smaller diameter. The present invention may be layered so that the water indicator is between the adhesive film and the desiccant paper. An opening may be cut into the middle or the circle making a ring. These components may be glued or taped together. Polyurethane plastic may then be shaped into a sleeve. The opening of the sleeve may be a similar size of the hole cut into the combined components above. The sleeve may be radiofrequency welded onto the non-tape/skin side of the above unit. The entire device may need to be created in a sterile environment.

As used in this application, the term “a” or “an” means “at least one” or “one or more.”

As used in this application, the term “about” or “approximately” refers to a range of values within plus or minus 10% of the specified number.

As used in this application, the term “substantially” means that the actual value is within about 10% of the actual desired value, particularly within about 5% of the actual desired value and especially within about 1% of the actual desired value of any variable, element or limit set forth herein.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

### INDUSTRIAL APPLICABILITY

Embodiments of the disclosed invention can be useful for protecting a catheter on a user.

What is claimed is:

1. A central venous catheter covering device comprising:
a sleeve formed of a water resistant and breathable polymer and comprising a first end and a second end, wherein the first end comprises a rim forming an opening into the sleeve;
an adhesive film comprising a first side and a second side, wherein the first side is attached to the rim of the sleeve and the second side comprises an adhesive, wherein the adhesive film comprises an inner edge forming an opening leading into the sleeve;
a water indicator comprising a ring shape having a first side, a second side, an inner edge and an outer edge, wherein the first side of the water indicator is attached to the second side of the adhesive film, wherein an opening formed by the inner edge aligns with the opening of the adhesive film and leads into the sleeve, wherein an outer edge of the adhesive film extends beyond the outer edge of the water indicator so that the adhesive is exposed;
a desiccant paper attached to the water indicator, wherein the water indicator is sandwiched in between the adhesive film and the desiccant paper;
a tab joined to the sleeve; and
a first adhesive strip joined to the tab and the sleeve and configured to be attached to skin of a human user.

2. The catheter covering device of claim 1, further comprising a second adhesive strip, joined to the tab and the sleeve, and linearly aligned with the tab and the first adhesive strip and configured to be attached to the skin of the human user.

3. The catheter covering device of claim 1, wherein the water indicator changes color when the water indicator is wet.

4. The catheter covering device of claim 1, further comprising a tab extending from the perimeter of the adhesive film.

5. The catheter covering device of claim 1, wherein the polymer is polyurethane.
